(19) United States
(12) Patent Application Publication
(10) Pub. No.: US 2007/0167972 A1
(43) Pub. Date: Jul. 19, 2007

(54) Balloon apparatus and methods

(76) Inventors: Charles Louis Euteneuer, St. Michael, MN (US); Duane G. Frion, Brooklyn Center, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/333,694

(22) Filed: Jan. 17, 2006

(51) Int. Cl. A61M 29/00

(52) U.S. Cl. 606/192

(57) ABSTRACT

Balloon apparatus to access bodily lumen of a patient are disclosed. The balloon apparatus include an inflation tube, a core wire and a balloon. The balloon is secured to a distal end of the inflation tube and a core wire extending from the distal end of the inflation tube passes through a sleeve in the balloon. A proximal tube may be provided at the proximal end of the inflation tube. The balloon apparatus may be configured with a relatively small diameter using aspects of the present inventions.

### BACKGROUND OF THE INVENTION

1. Summary of the Invention

The present inventions relate to medical devices and, more particularly, balloons for medical catheters and medical guidewires.

2. Description of the Related Art

Medical catheters and guidewires can be useful tools in treating intravascular disorders, disorders within other lumen of the body, extracting fluids from lumen as well as introducing fluid into lumen. Some medical device are configured to receive a medical catheter or guidewire to permit the medical device to permit the medical device to be positioned within the body of a patient. Most medical devices are configured to receive a medical catheter or guidewire having an outside diameter of around 0.014 inches. Further, many catheters and some guidewire designs can permit the inclusion of a balloon at or near the distal end of the catheter or guidewire. Depending on the configuration, these devices can also be used to introduce and/or expand various other medical devices, such as stents for example. A balloon may help to direct the distal end of the catheter through a lumen where the pulsatile flow of blood may the balloon to act as a “sail.” Further, the balloons in various configurations may be used to test for the occlusion of vessels, for embolization of bleeding, to treat or control vasospasms, and for treatment of nosebleeds, among other uses. In particular, silicone balloons have been shown to be effective for treating vasospasms.

Medical catheters and guidewires are particularly useful in accessing remote and tortuous locations within the body. Because of the need to navigate through the body to remote locations through narrow twisting lumen, medical catheters and guidewires are frequently long thin tubular devices. The materials and configurations of the tubular devices frequently warrant the positioning of a core wire within at least a portion of the catheter or guidewire to achieve the desired pushability and pushability for the devices. These core wires may extend through the central lumen of the catheter or guidewires. The central lumen is also frequently required to communicate of fluids to or from the distal portion of the medical catheter or guidewire. When a core wire is used, the core wire and lumen must be cooperate to permit the adequate flow of fluids and while maintaining the desired torquability and pushability for the device as a whole. Accordingly, a need exists for core wire and lumen configurations which permit the communication of fluids while maintaining the desired performance characteristics.

Further, there are significant benefits in reducing the diameter of medical catheters and guidewires for many applications. The reduced size is generally less traumatic to a patient. The reduced size also permits access to locations of reduced size or diameter that may not be reachable or treatable by larger diameter medical catheters and guidewires. Various locations in the brain or heart can be particularly difficult to reach and/or treat when the area to be treated or otherwise accessed is in the. This is, at least in part, due to the tortuous path that must be navigated to get to some locations as well as the point for introduction of the medical catheter or guidewire frequently being the femoral artery. Reduced diameter medical catheters and guidewires may have physical characteristics that may enhance the difficulty of placing them at such remote locations. Accordingly, a need exists for components that provide performance characteristics that simplify the placement of reduced diameter medical catheters and guidewires.

Manufacturing medical devices can be difficult. As the size of the devices decreases, the difficulty in manufacturing the devices generally increases. Medical catheters and guidewires have outside diameters as small as about 0.010 inches. Accordingly, a need exists for configurations of components that provide for simplified manufacture of reduced diameter medical catheters and guidewires.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

This present invention provides a balloon guidewire in the form of an elongated hollow tube having a balloon secured to its distal end. In one aspect, a balloon guidewire in accordance with the present invention may be formed from a single inflation tube defining an inflation lumen. In another aspect, a balloon guidewire in accordance with the present invention may be formed from one or more tubes defining a continuous passage. At least the inflation tube includes a core wire extending over at least a portion of the inflation lumen. At least a portion of the core wire extends beyond the distal end of the inflation tube and through the balloon secured to the distal end of the inflation tube. The balloon includes a sleeve which slidably receives the core wire. The sleeve may be integral with the balloon or may be a separate component secured to the balloon. Small bodily lumens can be accessed by the balloon guidewire and occluded using the balloon to provide certain types of diagnosis and/or treatment at the desired location within the body while also enabling access of over-the-wire instruments. A small outer diameter of the balloon guidewire can enable over-the-wire instruments to be used in conjunction with the balloon guidewire. The tubes may be formed by extrusion and drawing and typically have a sufficiently stiff proximal end, a flexible, atraumatic distal end, and a wall thickness to optimize the cross-sectional area of the lumen for particular applications. In one aspect, small bodily lumens of a patient can be accessed with a balloon guidewire in accordance with the present invention, by conventional guidewire techniques.

The proximal tube and distal tube may be made from various polymers, metals or composite materials. In one aspect, the proximal tube and/or the inflation tube can be formed from stainless steel. In another aspect, the proximal tube and/or the inflation tube can be formed from nitinol. One or both of the proximal tube and the inflation tube may be annealed progressively to vary the flexibility along the length of the proximal and the inflation tubes. The distal portions of the proximal tube may be annealed such that the distal portions has greater flexibility than proximal portions of the inflation tube.

In one aspect, the balloon guidewire can be constructed for insertion into the body to occlude blood flow in an artery. The balloon guidewire having a proximal tube extending a proximal portion of the length of the balloon guidewire, and an inflation tube secured to the proximal tube. The proximal tube may have a proximal lumen extending over at least a portion of the length of the proximal tube. The inflation tube has an inflation lumen extending over at least a portion of the length of the inflation tube. The inflation tube includes a distal end having a distal inflation tube opening to permit the communication of inflation media from the inflation lumen into the inflation chamber of the balloon. The distal inflation tube opening may be along the longitudinal axis or may be peripheral openings such as slots, holes or other openings in the inflation tube that are in fluid communication with the inflation chamber of the balloon.

The core wire extends from the distal end of the inflation tube. The core wire may have a round transverse cross-section and typically includes an atraumatic tip at its distal end. The balloon is secured about the distal end of the inflation tube or may be otherwise secured to the distal end of the inflation tube. The balloon forms an inflation chamber which extends around the distal end of the inflation tube. The balloon includes a sleeve which is typically positioned at the distal end of the balloon. The sleeve receives a portion of the length of the core wire extending from the distal end of the inflation lumen. The sleeve generally forms a seal about the core wire to prevent medically significant seepage of inflation media from the inflation chamber as the sleeve slides distally along the core wire during inflation and proximally along the core wire during deflation.

When the balloon guidewire includes a proximal tube and an inflation tube, the distal end of the proximal tube is secured to the proximal end of the inflation tube. If the proximal tube has a proximal lumen, the proximal lumen may be in fluid communication with the inflation lumen. In one aspect, a distal notch may be formed at the distal end of the proximal tube and a proximal notch may be formed at the proximal end of the inflation tube. The notches may be integrally formed with the tubes, may be formed by cutting or grinding, or may be otherwise formed. The proximal notch and the distal notch can be overlapped and secured to one another using adhesives, welding or other techniques.

An atraumatic tip secured to the distal end of the inflation tube may have a hemi-spherical or rounded tip for atraumatic insertion into the body. The atraumatic tip may be fabricated from a metal or may be a polymeric material such as PET, polyimide, or polyethylene. The atraumatic tip may include a shaping wire secured to or within the distal end of the inflation tube. A coil may extend around the shaping wire. The rounded tip may be secured to one or both of the coil and the shaping wire. The atraumatic tip may include one more components containing a radio-opaque material.

In another aspect, the present inventions feature methods of treating a patient using the balloon apparatus. The balloon apparatus is inserted into a body lumen and guided to a target location in the lumen requiring treatment. Once positioned at the target location, the balloon may be inflated. Inflation media is passed through the inflation tube and is directed into the balloon through the distal inflation tube opening. When the balloon apparatus is appropriately sized, an over-the-wire medical device may be slid in guided contact over the balloon apparatus to access the desired location such that a surgical, therapeutic or diagnostic procedure using the over-the-wire medical device may be performed. After the surgical, therapeutic or diagnostic procedure, the balloon may be deflated by withdrawing inflation media through the inflation lumen. The balloon apparatus may then be removed from the bodily lumen. In other aspects, the balloon apparatus may be removed from the over-the-wire medical device and bodily lumen prior to the surgical, therapeutic or diagnostic procedure.

Other features and advantages of the invention will become apparent from the following detailed description, and from the claims.

### BRIEF DESCRIPTION OF THE DRAWINGS

FIG. 1 illustrates a partial perspective view of an exemplary embodiment of a balloon guidewire in accordance with the present inventions;

FIG. 2 illustrates a detailed partial perspective view of an exemplary embodiment of a distal end of a balloon guidewire in accordance with the present inventions;

FIG. 3A illustrates a cross-section of a partial side view of an exemplary embodiment of a distal end of a balloon guidewire with an un-inflated balloon in accordance with the present inventions;

FIG. 3B illustrates a cross-section of a partial side view of an embodiment of a distal end of a balloon guidewire similar to the embodiment of FIG. 3A with a partially inflated balloon in accordance with the present inventions;

FIG. 3C illustrates a cross-section of a partial side view of an embodiment of a distal end of a balloon guidewire similar to the embodiment of FIG. 3A with a fully inflated balloon in accordance with the present inventions;

FIG. 4 illustrates a detailed partial perspective view of an exemplary embodiment of a distal end of a balloon guidewire in accordance with the present inventions;

FIG. 5A illustrates a cross-section of a partial side view of another exemplary embodiment of a distal end of a balloon guidewire with an un-inflated balloon in accordance with the present inventions;

FIG. 5B illustrates a cross-section of a partial side view of an embodiment of a distal end of a balloon guidewire similar to the embodiment of FIG. 5A with a partially inflated balloon in accordance with the present inventions;

FIG. 5C illustrates a cross-section of a partial side view of an embodiment of a distal end of a balloon guidewire similar to the embodiment of FIG. 5A and 5B with a substantially fully inflated balloon in accordance with the present inventions;

FIG. 6 illustrates a partial perspective view of an exemplary embodiment of a core wire in accordance with the present inventions;

FIG. 7 illustrates a cross-section of a partial side view of an exemplary embodiment of a core wire in accordance with the present inventions;

FIG. 8A illustrates a cross-sectional end view through section 8A-8A of FIG. 7; and

FIG. 8B illustrates a cross-sectional end view through section 8B-8B of FIG. 7.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

The present inventions provide balloon apparatus 10 and associated methods for use in conjunction with medical catheters and medical guidewires. The balloon apparatus 10 includes an inflation tube 14, a core wire 16 and a balloon 18. The balloon 18 is secured over an end of the inflation tube 14 to permit the introduction of inflation media into the balloon when the balloon is positioned within a patient. The core wire 16 is positioned within the inflation tube 14. The core wire may confer desired performance characteristics such as a desired torquability or a desired pushability to the balloon apparatus 10. The core wire extends through the end of the inflation tube 14 which includes the balloon 18 and passes through the balloon. The balloon includes a sleeve 38 that is slidably received over the guidewire. The sleeve 38 forms a seal to permit the inflation of balloon 18 and is typically configured to prevent medically significant amounts of inflation media from leaking from between the core wire 16 and the sleeve 38. The balloon apparatus 10 is generally configured to be introduced into a bodily lumen of a patient and have the balloon 18 positioned at a target location in the bodily lumen. Portions of the core wire 16 may further include one or more flattened portions 26 along their length to enhance the fluid flow through the inflation lumen 24 of the inflation tube 14 to or from the balloon 18 during inflation and deflation, respectively. The fluids typically introduced into the balloon 18 include various types of inflation media. The inflation media will frequently include various imaging compounds and may include various medicinal or other compounds that can be desirable in particular applications.

The Figures generally illustrate various embodiments of balloon apparatus 10 including aspects of the present inventions. The particular exemplary embodiments of the balloon apparatus 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, variations of balloon apparatus 10 for use with medical guidewires and medical catheters different from the illustrated embodiments may be encompassed by the appended claims.

As generally illustrated throughout the Figures, balloon apparatus 10 generally include an inflation tube 14, a core wire 16 and a balloon 18. The balloon apparatus 10 may further include a proximal tube 12. The inflation tube 14 has an outer surface 64 and an inner surface 74. The inner surface 74 defines an inflation lumen 24. A first inflation tube opening 34 in fluid communication with the inflation lumen 24 may be located at or about the proximal end 114 of the inflation tube 14. A distal inflation tube opening 44 in fluid communication with the inflation lumen 24 is located at the distal end 214 of the inflation tube 14. The balloon 18 is typically secured to the distal end 214 of inflation tube 14 such that the distal inflation tube opening 44 is in fluid communication with an inflation chamber 28 defined by balloon 18. In operation, the inflation lumen 24 may communicate inflation media from the proximal end 114 to a distal end 214 of the inflation tube 14 and into the inflation chamber 28 of balloon 18 through the distal inflation tube opening 44 to inflate balloon 18. The core wire 16 is typically received within and extends through the inflation lumen 24. In one aspect, the core wire is secured to the inflation tube 14 at one or more locations along its length. In other aspects, the core wire 16 may be secured in a lumen or other structure independent from the inflation lumen 24. Regardless of the configuration within the inflation tube 14, the core wire 16 extends from the inflation tube 14 into the inflation chamber 28 of the balloon 18. The balloon 18 includes a sleeve 38 through which the core wire 16 passes. The sleeve 38 may be integral with the balloon 18 or a separate structure secured to the balloon 18. As the balloon 18 inflates, sleeve 38 typically slides distally along a portion of core wire 16 or associated structure. For deflation, the inflation lumen 24 receives fluid from the balloon 18. As the balloon 18 deflates, the sleeve 38 typically slides proximally along a portion of the core wire 16 or associated structure. When included, a proximal lumen 22 of the proximal tube 12 is typically in fluid communication with the inflation lumen 24. For purposes of description, balloon apparatus 10 generally should be considered to have longitudinal axis 300 defined along its length as generally illustrated in the Figures regardless of any curvature in the balloon apparatus 10.

When present, the proximal tube 12 is secured to the proximal end of the inflation tube 14. To secure the inflation tube 14 to the proximal tube 12, the inflation tube 14 may define a distal notch 62 to receive a proximal notch 52 of the proximal tube 12. For purposes of the present inventions, the term “secured to” means that the distal tubular portion is attached to main proximal portion by a suitable method such as, for example, by welding, brazing, heat shrinking, or gluing among other methods.

The proximal tube 12 is generally configured to guide and position portions of the inflation tube 14 within a patient. The proximal tube 12 may function as the point of articulation for a user as the inflation tube 14 is introduced into a bodily lumen of a patient. The proximal lumen 22 of the proximal tube 12 extends over at least a portion of the length of the proximal tube 12. The proximal lumen 22 of the proximal tube 12 may extend longitudinally within the proximal tube 12 from a first proximal tube opening 32 to a second proximal tube opening 42 defined by the proximal tube 12. The proximal lumen 22 is generally configured to receive a fluid, such as, for example, inflation media, and communicate the fluid at least to the second proximal tube opening 32. The proximal tube lumen 22 is typically in fluid communication with the inflation lumen 24 of the inflation tube 14.

The proximal tube 12 may further define a proximal notch 52 at a distal end 212 of the proximal tube 12 which is configured to be secured to a distal notch 52 of the inflation tube 14. The proximal notch 52 generally extends from the distal end 212 of the proximal tube 12 to a location along the proximal tube 12 which is proximal to the distal end 212 of the proximal tube 12. The proximal notch 52 is generally shaped to receive the distal notch 62 at a first end 214 of inflation tube 14. The proximal notch 52 may extend into the proximal lumen 22. When the proximal tube 12 is secured to the inflation tube 14, the proximal lumen 22 and the inflation lumen 24 together may form a continuous passage extending between a proximal end 112 of proximal tube 12 and a distal inflation tube opening 44 of inflation tube 14.

The proximal tube 12 may be made from a variety of materials including polymers, metals, and various composite materials. In one aspect, the proximal tube 12 is made of a stainless steel. In another aspect, the proximal tube 12 is made of nitinol. Typically, the proximal tube 12 is configured to have a desired elastic range. The proximal tube 12 may be configured to have a desired balance of longitudinal stiffness and torsional rigidity based on the characteristics of the inflation tube 14. The longitudinal stiffness, at least in part, dictates the push characteristics for the proximal tube 12. The torsional rigidity, at least in part, dictates the precision of the rotational control provided by the proximal tube 12.

The proximal tube 12 may have various outside diameters 312 and lengths depending on the particular application for the balloon apparatus 10. Generally, the proximal tube 12 is configured to at least support inflation of the associated balloon 18. The particular configuration of proximal tube 12 may also depend upon whether or not the proximal tube 12 is intended primarily for use as a balloon catheter or as a wire support for other guidewires or catheters. For use primarily as a balloon catheter, a balloon apparatus 10 may be configured to support larger volumes of fluid than when the balloon apparatus 10 used as a delivery rail for other devices. In such applications for balloon inflation, the proximal tube 12 may have an outside diameter 312 of about 0.024 inches and a lumen diameter of about 0.019 inches. This outside diameter 312 can provide the desired torsional rigidity without being too longitudinally stiff. The inside diameter 412 of the proximal lumen 22 may be selected to provide a desire inflation/deflation time. For intercranial applications where the insertion point is in the femoral artery, the length of the proximal tube 12 can be about 110 centimeters. A proximal tube 12 of this length may keep the proximal tube 12 in the straight portion of the guide. For use of balloon apparatus 10 as a guide wire, the outside diameter 312 can be around 0.014 inches. For other applications requiring access to smaller bodily lumen, an outside diameter 312 of less than 0.014 inches may be used. In one exemplary embodiment, the proximal tube 12 of the balloon apparatus 10 can have an outside diameter 312 of the order of 0.014 inches and a wall thickness of the order of 0.002 inches to maximize the inside diameter 412 of the proximal lumen 22. The proximal tube 12 can be between about 165 cm to about 205 cm in length and although flexible, have a stiffness of about 50-100 N-mm₂ to impart sufficient lateral stiffness and torque transmission capabilities along its length.

The inflation tube 14 may be used without an associated proximal tube 12 or may be secured to the distal end 214 of a proximal tube 12. The inflation tube 14 defines at least one inflation lumen 24 to permit the communication of fluids along at least a portion of the length of the inflation tube 14. The inflation tube 14 is generally configured to facilitate the positioning of the distal end 114 of inflation tube 14 at a desired location within a bodily lumen of a patient as well as to permit the inflation of a balloon 18. When balloon apparatus 10 includes a proximal tube 12, the proximal end 114 of the inflation tube 14 is typically secured to a distal end 212 of the proximal tube 12. The inflation tube 14 includes a balloon 18 secured at or near the distal end 214 of the inflation tube 14. The inflation tube 14 further includes a core wire 16 extending within the inflation tube 14 over at least a portion of its length. The inflation tube 14 is generally configured to guide and position the balloon 18 within a bodily lumen of a patient.

The inflation lumen 24 of the inflation tube 14 extends over at least a portion of the length of the inflation tube 14. The inflation lumen 24 of inflation tube 14 may extend longitudinally within the inflation tube 14 from a first inflation tube opening 34 to a distal inflation tube opening 44 defined by the inflation tube 14. The inflation lumen 24 is generally configured to communicate a fluid along a portion of the length of the inflation tube 14. The inflation lumen 24 may receive a fluid, such as for example inflation media, at a location along its length and communicate the fluid to at least the distal inflation tube opening 44. In one aspect, the inflation lumen 24 may be configured to communicate a fluid from a proximal end 114 of inflation tube 14 to a distal end 114 of the inflation tube 14 or to a location adjacent to the distal end 214 of inflation tube 14. The inflation lumen 24 is typically in fluid communication with the proximal lumen 22 of the proximal tube 12 when a proximal tube 12 is included in the balloon apparatus 10.

The inflation tube 14 may be made from a range of materials and configurations depending upon the intended use for the resultant balloon apparatus 10. In one aspect, the tube may be a metal, such as, for example, stainless steel or nitinol. In another aspect, the inflation tube 14 can be made from one or more polymers such as polyethylene, nylon, polyimide, among others. The materials are generally selected to provide a desired balance of longitudinal stiffness and torsional rigidity based on the characteristics of the inflation tube 14 in combination with a core wire 16 extending along at least a portion of the length of the inflation tube 14.

The inflation tube 14 typically has an outside diameter 314 which is the same or smaller than the outside diameter of the proximal tube 12. For use primarily as a balloon catheter, the inflation tube 14 may have an outside diameter 314 of 0.024 inches. The inflation lumen 24 may then have a inside diameter 414 of about 0.020 inches. The inflation lumen 24 may be configured with as large a cross-sectional area as large as possible given the size and application for the balloon apparatus 10. In one exemplary embodiment, the inflation tube 14 of a balloon apparatus 10 has a length from about 15 cm to about 25 cm. The inflation tube 14 has an outside diameter 314 of about 0.014 inches and is secured to a proximal tube 12 having the same outside diameter 312. Inflation tube 14 may have a stiffness of about 25-50 N-mm² or less, to impart the desired flexibility to balloon apparatus 10. Additionally, the flexibility of balloon apparatus 10 may be varied by progressively annealing either a portion, for example, only inflation tube 14, or the entire length of balloon apparatus 10.

The core wire 16 is positioned within the inflation tube 14 and typically extends over at least a portion of the length of the inflation tube 14. The core wire 16 may be secured to or extend into the proximal tube 12 when a proximal tube 12 is included in the balloon apparatus 10. The core wire 16 may confer a desired balance of longitudinal stiffness and torsional rigidity characteristics to the inflation tube 14 through which the core wire 16 extends. Further, when secured to the proximal tube 12 the core wire 16 may transmit the torquing and pushing of the proximal tube 12 by a user to at least the distal portions of the inflation tube 14. In other aspects, the core wire 16 may be used to, at least in part, secure the distal tube 16 to the proximal tube 12 of a catheter 10. When secured along a length of the proximal tube 12, the core wire 16 may confer a desired balance of longitudinal stiffness and torsional rigidity characteristics to the portion of the proximal tube 12 through which the core wire 16 extends.

A portion of the core wire 16 extends distally from the distal end 212 of proximal tube 12 and along at least a portion of the length of inflation tube 14. The core wire is typically secured within the inflation tube 14 and may be secured within the inflation lumen 24 of the distal tube 14. In one aspect, the core wire 16 may be secured at one or more discrete locations along the length of the core wire 16. In other aspects, the core wire 16 may be rotatably and/or slidably received within the inflation lumen 24 of the inflation tube 14. When the balloon apparatus includes a proximal tube 12, the core wire 16 may be secured within the proximal tube 12. In one aspect, the core wire 16 may extend into and be secured within the proximal lumen 22 of the proximal tube 12. In one aspect, a distal end 216 of the core wire 16 may be secured to a proximal end 190 of an atraumatic tip 90.

The core wire 16 is typically a metal wire having a circular transverse cross-section as shown in FIG. 8A for exemplary purposes. The core wire 16 is typically made of a rigid but elastic material. Although the core wire 16 is typically made from stainless steel or nitinol, the core wire 16 may be formed from other metals, polymers or composite materials as will be recognized by those skilled in the art upon review of the present disclosure. The core wire 16 is typically a solid wire, however the core wire 16 may be hollow along at least a portion of its length. The core wire 16 may also be in the form of a wound cable, a braided filament, or otherwise alternatively configured as will be recognized by those skilled in the art upon review of the present disclosure. In other aspects, the core wire 16 may be tapered along the distal portion of the core wire such that the decreasing diameter provides greater flexibility to the region of the core wire 16 extending beyond the distal end 214 of the inflation tube 14. As illustrated for exemplary purposes throughout the Figures, the taper may be stepwise toward the distal end 216 of the core wire 16.

The core wire 16 may also include one or more reduced profile regions 28 as illustrated in FIGS. 8B in transverse cross-section as a portion of an arc of a circle for exemplary purposes. In other aspect, the transverse cross-section of the reduced profile regions may be semi-circular or otherwise shaped. The reduced profile regions 28 are flanked by regions of the core wire 16 having a circular transverse cross-section. The reduced profile regions 28 may provide a flow path or enlarged flow path relative to the flow path provided around the flanking portions of the core wire 16 having a circular transverse cross-section. These reduced profile regions 28 may provide for decreased localized and/or overall resistance to the flow of inflation media through the inflation lumen 24 including a core wire 16.

For intercranial applications, the core wire 16 may be about 40 centimeters long when the insertion point is the femoral artery. In an exemplary embodiment where the proximal tube 12 has an outside diameter 312 of 0.014 inches, the proximal end 116 of the core wire 16 can have a diameter 316 of about 0.009 inches where it attaches to the proximal tube 12. The core wire 16 may include several reductions in outside diameter 316 toward the distal end 216 of core wire 16. In this aspect, the core wire 16 may have a diameter of about 0.004 inches at the distal end 216 of the core wire 16.

A balloon 18 may be provided at or near the distal end 214 of the inflation tube 14 for inflation within the bodily lumen of a patient. In one aspect, a proximal end 118 of a balloon 18 may be positioned at or near the distal end 214 of the inflation tube 14. The balloon 18 defines an inflation chamber 28 to receive inflation media from the inflation lumen 24 of the inflation tube 14. Accordingly, the inflation chamber 28 is in fluid communication with the inflation lumen 24. Typically, the inflation chamber 28 is in fluid communication with at least one distal inflation tube opening 44. In one aspect, the balloon 18 may be positioned over at least one distal inflation tube opening 44 which is in fluid communication with the inflation lumen 24.

The balloon 18 includes a sleeve 38 that is slidably received over the core wire 16. The sleeve 38 forms a seal to permit the inflation of balloon 18 and is typically configured to prevent medically significant amounts of inflation media from leaking from between the core wire 16 and the sleeve 38. The sleeve 38 defines a sleeve passage 58 to receive a portion of the core wire 16. The core wire 16 extends from the inflation tube 14 into the inflation chamber 28 of the balloon 18 and through the sleeve passage 58 of the sleeve 38. The sleeve passage 58 typically has a shape which corresponds to the cross-sectional shape of the core wire 16 at the region of the core wire 16 passing through the sleeve 38. As the balloon 18 inflates, sleeve 38 typically slides distally along a portion of core wire 16 or associated structure. For deflation, the inflation lumen 24 receives fluid from the balloon 18. As the balloon 18 deflates, the sleeve 38 typically slides proximally along a portion of the core wire 16 or associated structure.

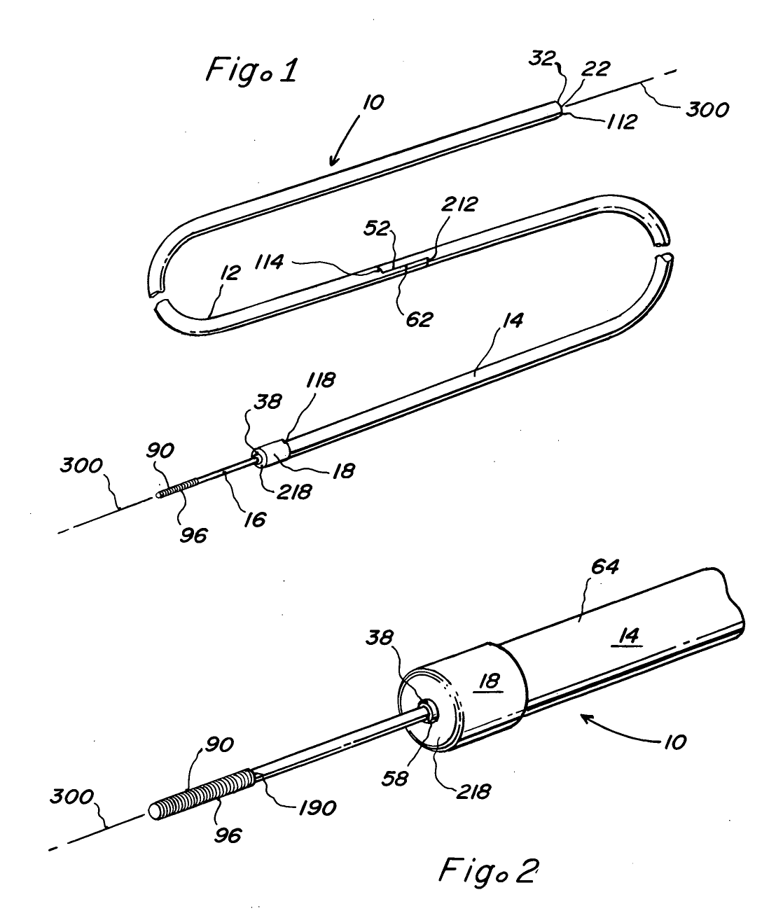

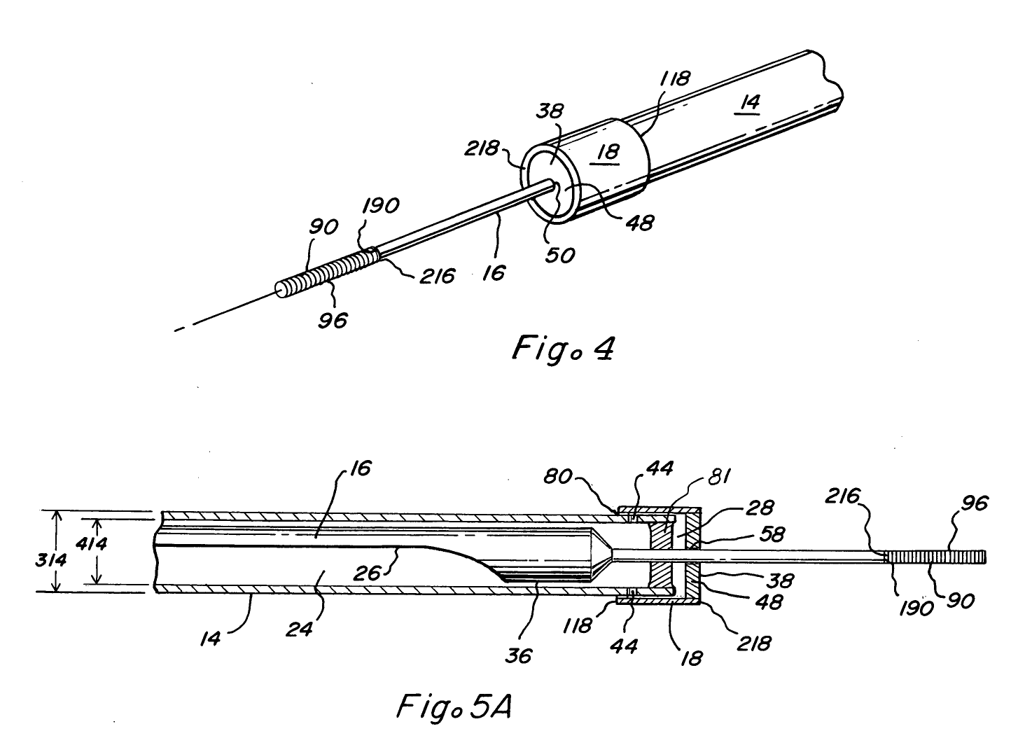

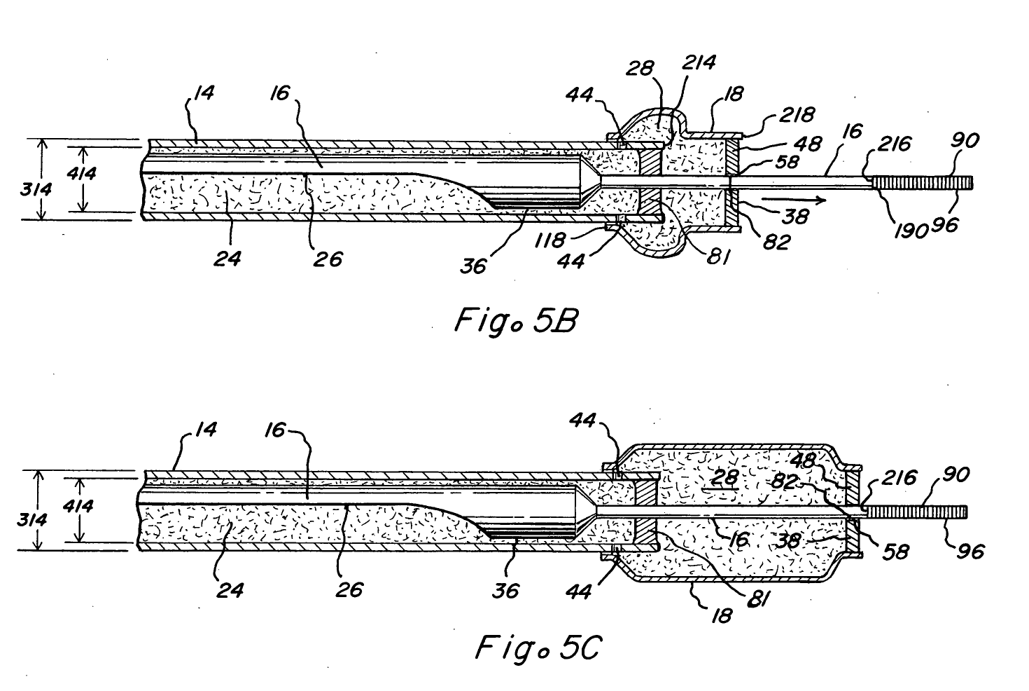

The sleeve 38 may be integral with the balloon 18, as illustrated in FIGS. 1 to 3C, for exemplary purposes, or a separate structure secured to the balloon 18, as illustrated in FIGS. 4 to 5C for exemplary purposes. When integral, the sleeve 38 may be a thickened or reinforced region of the balloon 18 that resists deformation and leaking upon introduction of inflation media into the expansion chamber 28 and inflation of the balloon 18. When a separate structure, the sleeve 38 may be a disk 48 for example, which defines the sleeve passage 58. The disk 48 may be peripherally secured to the balloon 18. The disk 48 may be generally expandable and elastic, it may be generally rigid, or it may be otherwise configured. However, the sleeve passage defined by the disk 48 is configured to resist deformation and leaking upon introduction of inflation media into the expansion chamber 28 and inflation of the balloon 18. A lubricious coating 82 may be provided between the sleeve 38 and the core wire 16 to reduce frictional forces between the sleeve 38 and core wire 16 during inflation and deflation as the sleeve 38 slides along the core wire 16. In one aspect, the lubricious coating 82 is provided over at least a portion of the sleeve passage 48. In another aspect, the lubricious coating 82 is provided over at least a portion of the length of the core wire 16.

Depending upon the application for the balloon apparatus 10, the balloon 18 may be configured with a wide range of physical specifications and performance characteristics as will be recognized by those skilled in the art upon review of the present disclosure. In one aspect, the balloon 18 may be either compliant or non-compliant. For various applications, the balloon 18 may be configured and sized to provide the desired inflated diameter and length for a treatment and location. In neurovascular applications, the target vessel diameters may range from as large as 10 to 12 millimeters to as small as 2 to 3 millimeters. The balloon 18 may be configured to circumferentially contact the walls of these vessels and may be provided in a variety of different lengths depending on the treatment and/or purpose of the balloon. In compliant embodiments, the balloon 18 may be made from silicone. For neurovascular applications, silicone may provide additional therapeutic benefits relating to spasms that will be recognized by those skilled in the art upon review of the present disclosure. When silicone is used, the silicone material may have a durometer of about 20 to 30. For neurovascular applications, this may give the balloon apparatus 10 the correct ‘feel’ when the balloon is inflated to a pressure of about 1 atmosphere.

An atraumatic tip 90 may be attached to the distal end 114 of the core wire 16. The atraumatic tip 90 generally provides a soft, gentle bumper for the distal end 216 of the core wire 16. The atraumatic tip 90 may include a coil 96. The coil 96 may be about 2 cm long and about 0.014 inches in diameter. The coil 96 can be made of 0.002 inches in diameter radio opaque material, preferably platinum. However, other materials known in the art can be used as well. A shaping ribbon may be positioned within the coil 96. The shaping ribbon is typically constructed from a metal and can serve several important functions. The shaping ribbon may serve as a bendable beam to more easily permit a user to induce a curved shape in the atraumatic tip 90 to direct the balloon apparatus 10 through a bodily lumen of a patient. Further, the shaping ribbon may improve the safety of a balloon apparatus 10 by not allowing the coils 96 of the atraumatic tip 90 to stretch out if a portion of the atraumatic tip 90 becomes lodged or otherwise hung up in the bodily lumen of a patient. The proximal end of the shaping ribbon may be attached to the distal end 216 of the core wire 16 and/or the proximal ends of the coils 96. The distal end of the shaping ribbon may be secured to the distal end of the coils 96. The thickness of the shaping ribbon for intercranial applications is typically about 0.002 inches by 0.004 inches. The shaping ribbon is made from a material having the desired combination of ductility and elasticity. Stainless steel of a proper temper is commonly used to provide these characteristics. The coil 96 may terminate in a rounded cap as to be generally atraumatic to the wall of a bodily lumen.

FIG. 1 and 2 illustrate an exemplary embodiment of an balloon apparatus 10 in accordance with the present inventions including both a proximal tube 12 and an inflation tube 14. FIG. 1 illustrates a general view of portions of the entire length of a balloon apparatus 10. FIG. 2 illustrate a more detailed view of a distal portion of a similar balloon apparatus 10. The proximal tube 12 and inflation tube 14 are illustrated as having a circular cross-section for exemplary purposes. The illustrated embodiment includes a passage extending from a proximal end 112 of the proximal tube 12 to a distal inflation tube opening 44 underlying the balloon 18 at a region proximal to the distal end 214 of the inflation tube 14 to communicate inflation media from the proximal end 112 of the proximal tube 12 to the inflation chamber 28 of the balloon 18. The passage is formed by connecting the proximal tube 12 to the inflation tube 14 such that the proximal lumen 22 of the proximal tube 12 is in fluid communication with the inflation lumen 24 of the inflation tube 14. As illustrated in FIG. 1, the proximal tube 12 is secured to the inflation tube by overlapping a proximal notch 52 in the proximal tube 12 with a distal notch 62 in the inflation tube 14. A distal portion of a core wire 16 is shown extending through a sleeve passage 58 of sleeve 38. An atraumatic tip 90 is shown secured to the distal end 216 of core wire 16. In the illustrated embodiment, the inflation tube 14 is generally configured to be directed through a bodily lumen within a patient by a physician manipulating the proximal tube 12 and, once properly positioned, to have the balloon 18 inflated for diagnostic or therapeutic purposes.

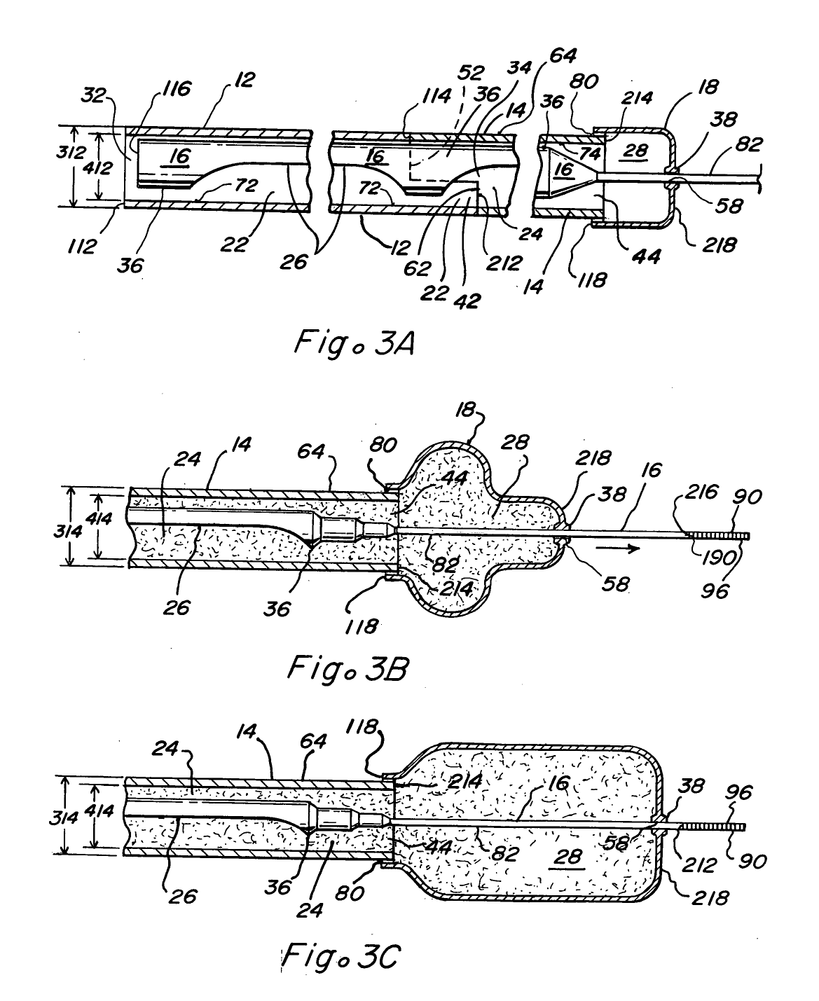

FIGS. 3A to 3C illustrate a cross-section of an exemplary embodiment of apparatus in accordance with the present invention. FIG. 3A shows exemplary cross-sections at the proximal end 112 of the proximal tube 12; the junction of the proximal tube 12 and the inflation tube 14; and the distal end 214 of the inflation tube 14 including balloon 18 in a substantially un-inflated configuration. FIGS. 3B and 3C show the distal end 214 of the inflation tube 12 including balloon 18 in a partially inflated and a substantially fully inflated configuration, respectively. As illustrated for exemplary purposes, the proximal tube 12 has a constant outside diameter 312 along its length and the inflation tube 14 has a constant outside diameter 314 along its length. Further, the outside diameter 312 and inside diameter 412 of the proximal tube 12 is illustrated as substantially the same as the outside diameter 314 and inside diameter 414 of the inflation tube 14 for exemplary purposes. In certain applications, varying outside diameters and inside diameters may be utilized to meet particular performance requirements.

FIG. 3A from left to right illustrates the proximal, intermediate, and distal portions of an exemplary embodiment of a balloon apparatus 10. The proximal portion includes the aspects of the proximal tube 12 and core wire 16. The proximal tube 12 includes an inner surface 72 defining the proximal lumen 22. The proximal lumen 22 extends from a first proximal tube opening 12 to a second proximal tube opening 42. The second proximal tube opening 42 is shown as peripherally secured to the proximal inflation tube opening 34 for purposes of exemplifying the junction between the proximal tube 12 and inflation tube 14 at proximal notch 52 and distal notch 62, respectively. The core wire 16 has a proximal end 116 positioned within the proximal tube and extends distally through the proximal lumen 22 and inflation lumen 24 and then out the distal end 214 of the inflation tube 14. The illustrated core wire 16 includes three circular profile regions 36 positioned between two reduced profile regions 26. The reduced profile region 26 provides a larger flow path to reduce resistance to fluid flow through the proximal lumen 22 and inflation lumen 24 during inflation and deflation of balloon 18. The distal end 212 of the proximal tube 12 is shown having a proximal notch 52 and the proximal end 114 of the inflation tube 14 is shown having a distal notch 62. The proximal notch 52 and the distal notch 62 are shown secured to one another at the junction of the proximal tube 12 and the inflation tube 14. As illustrated, the proximal notch 52 of the proximal tube 12 is overlapped with the distal notch 62 of the distal tube 14. The surfaces defining the notches are secured to one another to interconnect the proximal tube 12 and the distal tube 14. The surfaces of the proximal notches 52 and distal notch 62 may be welded, adhesively bonded, or otherwise secured to one another.

The distal end of FIG. 3A illustrates an exemplary balloon 18 having a proximal end 118 secured over the distal end 214 of the inflation tube 14. The proximal end 218 of the balloon 18 is in fluid conmmunication with the lumen 24 through a distal inflation tube opening 44 at the distal end 214 of the inflation tube 14. As illustrated for exemplary purposes, an adhesive 80 is used to secure the balloon 18 to the inflation tube 14. Welding, shrinking, expanding, mechanical bands, or other methods or devices may alternatively be used to secure the balloon 18 to the inflation tube 14. A portion of the core wire 16, shown tapered to a reduced diameter for exemplary purposes, extends into and through an inflation chamber 28 defined by the balloon 18 and passes through a sleeve passage 58 of sleeve 38.

As illustrated in FIGS. 3B and 3C, an atraumatic tip 90 is secured to the distal end 216 of core wire 16. The atraumatic tip includes a coil 96. As illustrated, the sleeve 38 is generally configured to allow the distal end 318 of balloon 18 to slide proximally and distally as the balloon 18 is inflated and deflated respectively. A lubricious coating 82 is provide on the core wire 16 for exemplary purposes in the embodiments of FIGS. 3A to 3C. FIG. 3B illustrates the balloon 18 in a of FIG. 3A in a partially inflated configuration. The inflation media introduced in the inflation chamber 28 through the inflation lumen 24 and distal inflation tube opening 44 is shown first inflating the proximal end 118 of the balloon 18. As the balloon 18 has inflated, the distal end 218 of the balloon has been displaced distally along the core wire 16 as the sleeve 38 slides along the core wire 16. FIG. 3C illustrates the balloon 18 of FIGS. 3A and 3B in a fully inflated configuration. The inflation media introduced in the inflation chamber 28 through the inflation lumen 24 and distal inflation tube opening 44 is shown having inflated the balloon 18 from the proximal end 118 to the distal end 218 of the balloon 18. In the illustrated balloon apparatus 10, the distal end 218 of the balloon is displaced distally along the core wire 16 to about the distal end 216 of the core wire 16 for exemplary purposes when the balloon 18 substantially fully inflated.

FIG. 4 illustrates the distal portion of another exemplary embodiment of an balloon apparatus 10 in accordance with the present inventions. The inflation tube 14 is again illustrated as having a circular cross-section for exemplary purposes. The illustrated embodiment may include a passage in the form of inflation lumen 24 extending from a first end 114 of the inflation tube 14 to a distal inflation tube opening 44 underlying the balloon 18 at a region proximal to the second end 214 of the inflation tube 14 to communicate inflation media from the proximal end 112 of the proximal tube 12 to the inflation chamber 28 of the balloon 18. A distal portion of a core wire 16 is shown extending through a sleeve passage 58 of sleeve 38 secured to balloon 18. The sleeve 38 is illustrated for exemplary purposes as a disk 48 defining an axially positioned sleeve passage 58. An atraumatic tip 90 is shown secured to the distal end 216 of core wire 16. In the illustrated embodiment, the inflation tube 14 is generally configured to be directed through a bodily lumen within a patient by a physician manipulating the proximal portion of the inflation tube 14 and, once properly positioned, to have the balloon 18 inflated for diagnostic or therapeutic purposes.

FIGS. 5A to 5C illustrate a cross-section of the distal portion of an exemplary embodiment in accordance with the present invention. FIGS. 5A to 5C show an exemplary cross-section at the distal end 214 of the inflation tube 14 including balloon 18 sequentially expanded from a substantially un-inflated configuration in FIG. 5A to a substantially fully-inflated configuration in FIG. 5C.

FIG. 5A illustrates an exemplary balloon 18 having a proximal end 118 secured over the distal end 214 of the inflation tube 14 with the balloon 18 in a substantially un-inflated configuration. The proximal end 118 of the balloon 18 is in fluid communication with the lumen 24 through a plurality of distal inflation tube openings 44 positioned proximal to the distal end 214 of the inflation tube 14. As illustrated for exemplary purposes, an adhesive 80 is used to secure the balloon 18 to the inflation tube 14. Welding, shrinking, expanding, mechanical bands, or other methods or devices may alternatively be used to secure the balloon 18 to the inflation tube 14. A region of the core wire 16 proximal to that extending from the distal end 214 of inflation tube 14 is secured within inflation tube 14 with a core wire adhesive 81. The core wire adhesive 81 is shown extending about the distal portion of the core wire 16 to secure the core wire 16 to the inflation tube 14 and, for exemplary purposes, sealing the inflation lumen 24 from expansion chamber 28 of balloon 18. Two proximally positioned distal inflation tube openings 44 are illustrated for exemplary purposes on diametrically opposite side of the inflation tube 14. As illustrated, the distal inflation tube openings 44 communicate inflation media into a proximal portion of the inflation chamber 28. A portion of the core wire 16, shown tapered to a reduced diameter for exemplary purposes, extends into and through an inflation chamber 28 defined by the balloon 18 and passes through a sleeve passage 58 of sleeve 38. The sleeve 38 is shown as a disk 48 peripherally secured to the balloon 18 to form the inflation chamber 28. As illustrated, the sleeve 38 is generally configured to allow the distal end 218 of balloon 18 to slide proximally and distally as the balloon 18 is inflated and deflated respectively. A lubricious coating 82 is provided on the sleeve 38 within the sleeve passage 58 for exemplary purposes.

FIG. 5B illustrates the balloon of FIG. 5A in a partially inflated configuration. The inflation media introduced in the inflation chamber 28 through the inflation lumen 24 and the plurality of distal inflation tube opening 44 is shown first inflating the proximal end 118 of the balloon 18. As the balloon 18 has inflated, the distal end 218 of the balloon has been displaced distally along the core wire 16 as the sleeve 38 slides along the core wire 16. FIG. 5C illustrates the balloon 18 of FIGS. 5A and 5B in a fully inflated configuration. The inflation media introduced in the inflation chamber 28 through the inflation lumen 24 and the plurality of distal inflation tube opening 44 is shown having inflated the balloon 18 from the proximal end 118 to the distal end 218 of the balloon 18. With the balloon fully inflated, the distal end 218 of the balloon has been displaced distally along the core wire 16 to about the location of atraumatic tip 90 at the distal end 216 of the core wire 16 for exemplary purposes. As inflation media is removed from the inflation chamber 28, the distal end 218 of the balloon 18 may move proximally along the core wire 16 until the balloon 18 is in a relaxed and deflated condition.

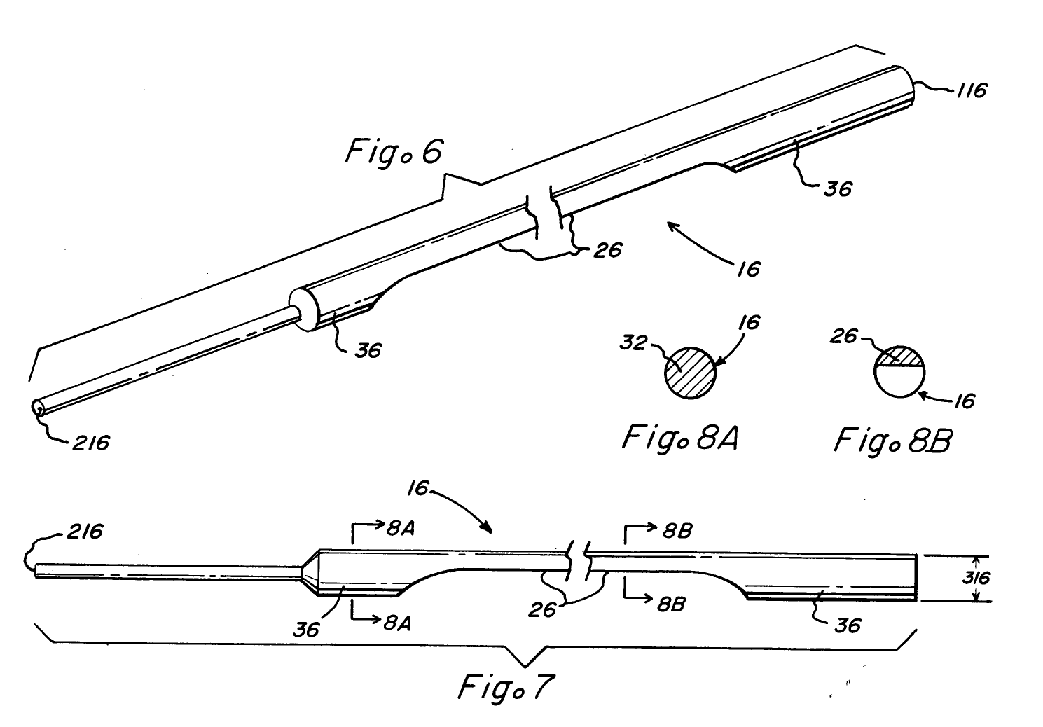

FIGS. 6 to 8B illustrate details of an exemplary core wire 16 in accordance with the present inventions. FIG. 6 provides a perspective view and FIG. 7 provides a side view of a core wire 16 having a reduced profile region 16 flanked proximally and distally by circular profile regions. The reduced profile region 26 may generally reduce the resistance to fluid flow through a proximal lumen 22 and/or inflation lumen 24 which include a core wire 16 extending therethrough. The distal portion includes a tapered region which provides a reduced diameter at the distal end 216 of the core wire 16. The core wire 16 may be secured at one or more locations along the inflation lumen 24 and, when present, the proximal lumen 22. FIG. 8A illustrates a cross-section through section lines 8A-8A of FIG. 7 showing an exemplary circular profile region 36 with a circular cross-sectional shape. FIG. 8B illustrates a cross-section through section lines 8B-8B of FIG. 7 showing an exemplary reduced profile region 26 having a transverse cross-sectional shape of a portion of an arc of a circle.

To use an balloon apparatus 10 in accordance with the present invention, a user may insert the distal end of balloon apparatus 10 into a bodily lumen of a patient using, for example, the Seldinger technique. The balloon apparatus 10 is guided through the bodily lumen to a location within the patient requiring treatment. As balloon apparatus 10 is guided through the patient, a user can manipulate the proximal tube 12 or the proximal end 114 of the inflation tube 14 to direct the distal end 214 of the inflation tube 14 through the bodily lumen. When the distal end 214 of the inflation tube 14 is positioned at or near the location within the bodily lumen requiring treatment, the user may initiate the desired treatment. In embodiments where the balloon apparatus 10 includes a balloon 18 at or near the distal end 214 of the inflation tube 14, the balloon 18 may be inflated to a desired size and/or pressure to affect the desired treatment. An balloon apparatus 10 including a balloon 18, properly sized and configured, may enable a user to access more distal or tortuous regions of the body. For example, when the distal portion of the balloon apparatus 10 has an outside diameter of around 0.014 inches, small lumen such as various arteries and veins in the brain and heart may be more easily accessed for diagnosis and/or treatment of the particular lumen or region.

Balloon apparatus 10 may further be used to guide surgical, therapeutic or diagnostic instruments over balloon apparatus 10 to access a desired location in a bodily lumen. When the instrument is positioned at the desired location within the bodily lumen, at least one surgical, therapeutic or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A balloon apparatus for accessing a bodily lumen of a patient, comprising:
an inflation tube having an outer surface and an inner surface, the inner surface defining an inflation lumen extending along at least a portion of the inflation tube, the inflation tube defining a distal inflation tube opening;
a core wire extending through at least a portion of the inflation tube and extending from the distal end of the inflation tube; and
a balloon defining an inflation chamber, the balloon secured over the distal portion of the inflation tube with the inflation chamber in fluid communication with distal inflation tube opening, the balloon including a sleeve, the core wire slidably received within the sleeve.

2. A balloon apparatus, as in claim 1, further comprising the sleeve defining a sleeve passage slidably receiving a portion of the core wire.

3. A balloon apparatus, as in claim 2, further comprising the sleeve integral with the balloon.

4. A balloon apparatus, as in claim 3, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.

5. A balloon apparatus, as in claim 4, the core wire further comprising at least one reduced profile region.

6. A balloon apparatus, as in claim 5, further comprising the reduced profile region have a transverse cross-sectional shape of a portion of an arc of a circle.

7. A balloon apparatus, as in claim 2, further comprising the sleeve secured to the balloon.

8. A balloon apparatus, as in claim 7, the sleeve comprising a disk peripherally secured to the balloon.

9. A balloon apparatus, as in claim 8, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.

10. A balloon apparatus, as in claim 9, the core wire further comprising at least one reduced profile region.

11. A balloon apparatus, as in claim 10, further comprising the reduced profile region have a transverse cross-sectional shape of a portion of an arc of a circle.
